(19) United States
(12) Patent Application Publication
(10) Pub. No.: US 2025/0349391 A1
(43) Pub. Date: Nov. 13, 2025

(54) ANALYSIS SYSTEM AND METHOD USING A MOLECULAR SENSOR

(71) Applicant: ATONARP INC., Minato-ku, Tokyo (JP)

(72) Inventors: Kazuo OKEMOTO, Tokyo (JP); Prakash Sreedhar MURTHY, Tokyo (JP)

(73) Assignee: ATONARP INC., Minato-ku, Tokyo (JP)

(21) Appl. No.: 18/870,522

(22) PCT Filed: May 30, 2023
(86) PCT No.: PCT/JP2023/019998

§ 371 Date: Nov. 29, 2024

(30) Foreign Application Priority Data
Jun. 1, 2022 (JP) 2022-089920

(51) Int. Cl. G16C 20/10; G01N 21/65; G16C 20/70; G16C 20/90

(52) U.S. Cl. CPC G16C 20/10; G01N 21/65; G16C 20/70; G16C 20/90

(57) ABSTRACT

An analysis system includes: a measurement apparatus that measures an object for analysis in a sample by a first measurement method; a database storing information relating to probes, tags, and linkers; a simulator that virtually synthesizes a molecular sensor including a tag, a probe, and a linker, and obtains a virtual measurement result of the first measurement method with the virtual molecular sensor; a molecular sensor providing apparatus that selects and provides a molecular sensor suited to analysis of the sample based on the virtual measurement results and preliminary measurement results of the sample by the measurement apparatus; and an analysis apparatus that analyzes components of the sample based on the measurement results of the sample by the measurement apparatus with the molecular sensor provided by the molecular sensor providing apparatus.

### TECHNICAL FIELD

The present invention relates to an analysis system and method using a molecular sensor.

### BACKGROUND ART

International Patent Publication WO2005/030996 discloses a method for producing and/or using molecular barcodes. Such barcodes include polymer backbones that may contain one or a plurality of branch structures. Tags may be attached to the backbone and/or branch structures. The barcode may also include a probe that can bind to a target, such as proteins, nucleic acids, and other biomolecules or aggregates. Different barcodes may be distinguished by the type and location of the tags. In other embodiments, barcode may be produced by hybridization of one or more tagged oligonucleotides to a template including a container section and a probe section. The tagged oligonucleotides may be designed as modular code sections to form different barcodes that are specific to different targets. In alternative embodiments, barcodes may be prepared by polymerization of monomeric units. Bound barcodes may be detected by various imaging modalities, such as surface plasmon resonance, fluorescent or Raman spectroscopy.

### SUMMARY OF INVENTION

Research is currently being performed into imaging and/or quantitative measurement of trace components using molecular sensors that are equipped with tags, such as molecular barcodes, that may be detected by methods such as Raman spectroscopy, a probe that binds to one or more target molecules, metals, or ions, and a linker (backbone, framework) that connects the tags and the probes. Measurement is often performed on multicomponent samples, and it is possible to combine tags, probes, and linkers in various ways according to the targets and detection methods. However, there are issues to be resolved, such as whether it is possible to apply a desired detection range (measurement range), for example, Raman tags that can be detected by Raman spectroscopy, for a measurement range (wavelength range) that can be detected by the respective measurement apparatuses (detection devices) such as standard Raman scattering, coherent anti-Stokes Raman spectroscopy (CARS), and stimulated Raman scattering (SRS), and whether it is possible to efficiently detect Raman tags by making use of the fingerprint region and the silent region of the main components.

One aspect of the present disclosure is an analysis system that uses one or more molecular sensors. This system includes: a measurement apparatus that is configured to measure an object for analysis (analyte) of a sample using a first measurement method; a database that stores information including structures and optical properties relating to a plurality of probes, a plurality of tags, and a plurality of linkers capable of connecting any of the plurality of probes and any of the plurality of tags; and a simulator that is configured to virtually synthesize a molecular sensor including (i) at least one tag that is able to be uniquely detected by the first measurement method out of the plurality of tags, (ii) an arbitrary probe, and (iii) a linker capable of connecting the at least one tag and the probe, and obtain a virtual measurement result of the first measurement method with the virtual molecular sensor synthesized virtually. The system further includes a molecular sensor providing apparatus that is configured to select and provide at least one molecular sensor including a probe, a tag, and a linker that are suited to analyzing multiple components including the target contained in the sample based on the preliminary measurement results of the sample by the measurement apparatus and virtual measurement results by the simulator. The at least one virtual molecular sensor includes (i) at least one probe for at least one of the components of the object in the sample as the target, (ii) at least one tag that is detectable at an expected concentration of the target and (iii) at least one linker capable of connecting the at least one probe and the at least one tag. The system further includes an analysis apparatus that is configured to analyze components of the sample based on measurement results of the sample by the measurement apparatus with the at least one molecular sensor provided by the molecular sensor providing apparatus.

This system makes it possible to provide a molecular sensor that considers the measurement method, the measurement range (or detection range, as one example, the frequency range of a spectrum), the main components and weak components contained in the object to be measured (in a sample), weak components or the like of interest to an application, and the like. The weak components may include components that are present in trace amounts or components that are difficult to measure by a specific measurement method (first measurement method). This means that even in the case of a sample that contains components that are difficult to measure with high accuracy or that cannot be measured or are difficult to measure using the first measurement method, the components of the sample can be analyzed via one or more molecular sensors and highly accurate analysis, even quantitative analysis, is likely to be performed.

The system may further include a learning apparatus that is configured to generate a learned model (trained learning model) that has been trained using learning data that includes replicas of a plurality of measurement results including the virtual measurement results of the simulator with the at least one molecular sensor provided by the molecular sensor providing apparatus as the virtual molecular sensor for a plurality of virtual samples with a plurality of virtual components which include the target and have different expected concentrations. The analysis apparatus may include an analysis module that analyzes a result produced by measurement using this learned model.

The system may further include an address assignment apparatus that is configured to assign a two-dimensional or three-dimensional address to the at least one molecular sensor provided from the molecular sensor providing apparatus. The measurement apparatus is configured to measure the sample in units of addresses using the first measurement method. As one example, a multi-flow cell or multi-address substrate may be used.

A typical first measurement method is Raman spectroscopy, for example CARS, and the tags may include Raman tags. The linker may include an organic molecular backbone (framework) and the molecular sensor may be a biosensor that uses biological materials (such as enzymes, antibodies, nucleic acids, and microorganisms) as probes to detect a target and produce a signal. The first measurement method may be any of standard Raman scattering, resonance Raman scattering, surface-enhanced resonance Raman scattering (SERS), tip-enhanced Raman scattering (TERS), coherent anti-Stokes Raman spectroscopy (CARS), stimulated Raman scattering (SAS), inverse Raman spectroscopy, stimulated gain Raman spectroscopy, hyper-Raman scattering, molecular optical laser examiner (MOLE), Raman microprobe, Raman microscopy, confocal Raman micro spectrometry, three-dimensional scanning Raman, Raman saturation spectroscopy, time-resolved resonance Raman, Raman decoupling spectroscopy, and UV-Raman microscopy.

Another aspect of the present invention is a method for performing analysis using a system including a measurement apparatus that measures an object for analysis (analyte) of a sample according to a first measurement method. The system includes: a database that stores information including structures and optical properties relating to a plurality of probes, a plurality of tags, and a plurality of linkers capable of connecting any of the plurality of probes and the plurality of tags; and a simulator that virtually synthesizes a molecular sensor including at least one tag that can be uniquely detected by the first measurement method out of the plurality of tags, an arbitrary probe, and a linker capable of connecting the tag and the probe, and obtains a virtual measurement result of the first measurement method with a virtual molecular sensor virtually synthesized. The method includes the following steps:
obtaining virtual measurement results by the simulator with at least one virtual molecular sensor including at least one probe for at least one of the components of the object contained in the sample as a target, at least one tag detectable at an expected concentration of the target, and at least one linker capable of connecting the probe and the tag;
selecting at least one molecular sensor which includes a probe, a tag, and a linker that are suited to analyzing multiple components including the target contained in the sample based on the virtual measurement results and preliminary measurement results of the sample by the measurement apparatus; and
analyzing components of the sample based on measurement results of the sample by the measurement apparatus with the at least one selected molecular sensor.

The method may further include the following steps:
generating replicas of a plurality of measurement results including the virtual measurement results of the simulator with the virtual molecular sensor, which is the at least one molecular sensor selected, for a plurality of virtual samples with a plurality of virtual components which include the target and have different expected concentrations; and
generating a learned model by performing machine learning with the replicas of the plurality of measurement results as learning data.

The step of analyzing may include analyzing the measurement results using the learned model.

The method may assign a two-dimensional or three-dimensional address to the selected at least one molecular sensor, and the analyzing may include mearing by the measurement apparatus the sample in units of addresses.

### BRIEF DESCRIPTION OF DRAWINGS

FIG. 1 is a block diagram depicting one example of an information providing system including an analysis system.

FIG. 2 depicts one example of a molecular sensor.

FIG. 3 depicts one example of detection by a molecular sensor.

FIG. 4 depicts examples of virtual measurement results.

FIG. 5 depicts one example of a programmable multi-address cell.

FIG. 6 is a flowchart depicting an overview of an analysis method using an analysis system.

### DESCRIPTION OF EMBODIMENTS

FIG. 1 depicts one example of an information providing system 1. In the information providing system 1, components of a sample 2, such as a liquid, are analyzed (identified) by measuring one or more objects for analysis (analytes) included in the sample 2, and an application 5 performs a predetermined operation or process, such as health management, diagnostic support and others. The system 1 includes a system 10 for measuring and analyzing a sample 2, which contains objects for analysis (analytes, substances to be analyzed, objects to be measured) via a light-transmitting holder 20 in which a predetermined amount (quantity) of the sample 2 is internally held or flows at a predetermined flow rate. The analysis system 10 is a system that enables weak components (trace components) contained in (included in) the sample 2 to be measured and analyzed with high accuracy using one or more molecular sensors 30. The fluid sample 2, which may include a liquid (including an aqueous solution or other solution) or a gas to be analyzed, may include a fluid used in a manufacturing process, a fluid discarded during a manufacturing process, atmospheric air, river water, wastewater, blood, serum, bodily fluid, a culture medium, an amplification medium, or the like. The application 5 may be manufactured and/or developed for various purposes, such as quality control, system monitoring, system control, environmental monitoring, health monitoring, diagnostic support, treatment monitoring, hazard monitoring, and the like. The sample 2 may include one or more components of interest (or region of interest) that is one item of information required by the application 5 to achieve its purpose, and one or more components (substances or specimens) to be analyzed (measured) by the analysis system 10 may be provided by the application 5 as components of interest 5a. The analysis result 10a of the analysis system 10 may be fed back to the application 5, and the application 5 can perform processing based on the analysis result 10a and/or provide information to a user or the like. As one example, the sample 2 may be a substance that is excreted from an organism, such as the human body. The sample 2 may be a urine sample, dialysis effluent, or exhaled air (exhaled gas).

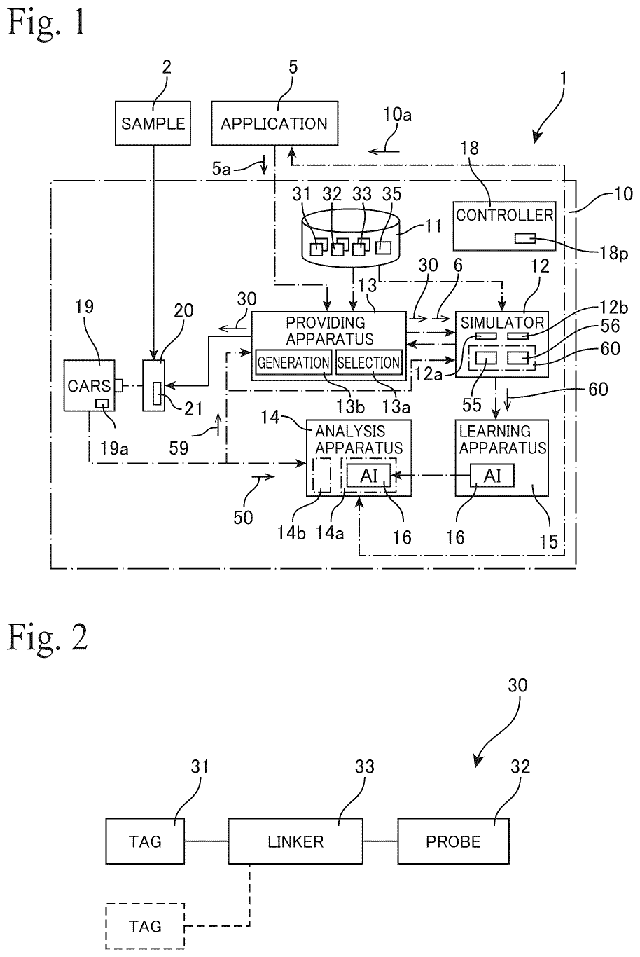

FIG. 2 depicts one example of the molecular sensor 30. The molecular sensor 30 is programmable and includes at least one tag 31 that can be uniquely detected by a predetermined measurement method (specific measurement method, first measurement method), a probe (affinity) 32 that targets and binds to a predetermined molecule, metal, or ion as a target, and a linker 33 that links the at least one tag 31 and the probe 32. One example of the molecular sensor 30 is a molecular barcode, one example of which is disclosed in International Patent Publication WO2005/030996 (Japanese Unexamined Patent Application Publication 2007-506431), the contents of which are incorporated herein by reference. The molecular sensor 30 may be made of an inorganic material. The linker may include an organic molecular backbone (framework), such as an organic polymer backbone. The molecular sensor 30 may be a biosensor that uses biological materials (enzymes, antibodies, nucleic acids, microorganisms, or the like) as the probe 32 to detect a target and produce a signal. The molecular sensor 30 may be hydrophobic or hydrophilic.

One example of a first measurement method is Raman spectroscopy, which as specific examples may be CARS (Coherent Anti-Stokes Raman Scattering), SRS (Stimulated Raman Scattering), time-resolved CARS, surface-enhanced resonance Raman scattering (SERS), or tip-enhanced Raman scattering (TERS), which are suitable for analysis of trace amounts. One example of a tag 31 that is uniquely detectable by Raman spectroscopy is a molecule (or molecular sequence) called a “Raman tag”, which can be created in a variety of ways, such as by repeating the same molecular sequence or attaching different groups so that a different spectrum is produced by each type of Raman spectroscopic analysis. The Raman tags 31 may be generated to produce unique spectra in a fingerprint region that is the spectral region of a main component contained in the sample 2, or may be generated to produce a unique spectrum in a silent region that does not include the fingerprint region. One example of the Raman tag 31 is a tag made of an alkyne and/or a nitrile. Examples of Raman tags are disclosed in “Raman and SERS Microscopy for Molecular Imaging of Live Cells” by Almar F Palonpon, Jun Ando, Hiroyuki Yamakoshi, Kosuke Dodo, Mikiko Sodeoka, Satoshi Kawata, and Katsumasa Fujita (Nature Protocols, Volume 8, pages 677-692 (2013)). Several examples of Raman tags are also disclosed in “Super-Multiplexed Optical Imaging and Barcoding with Engineered Polyynes” by Fanghao Hu, Chen Zeng, Rong Long, Yupeng Miao, Lu Wei, Qizhi Xu, and WeiMin (Nat Methods. Author manuscript; available in PMC 2018 Jul. 15).

The molecular sensor (molecular barcode) 30 may contain, as a tag 31, a polymeric Raman label attached to one or a plurality of probes for detecting target molecules. Some examples of polymeric Raman labels are disclosed in International Patent Publication WO2005/030996. A polymeric Raman label may contain 1 to 25 or even more Raman tags, and each individual Raman tag attached to a single polymeric Raman label may be different. Alternatively, a polymeric Raman label may contain two or more copies of the same Raman tag. A Raman tag 31 may be attached directly to the linker (backbone, framework) 33 or may be attached via a spacer molecule. Polymeric Raman labels (identifiers) are labels that can offer a wider variety of spectral differentiation than monomeric labels (monomeric identifiers), while still respecting the sensitivity of Raman spectroscopic detection.

One of example of probes 32 is affinity ligands, which are ligands that contain one of more capture molecules, here including any molecule that can bind to any of a plurality of target objects. Examples of capture molecules include, but are not limited to, antibodies, antibody fragments, genetically modified antibodies, single-chain antibodies, receptor proteins, binding proteins, enzymes, inhibitor proteins, lectins, cell adhesion proteins, oligonucleotides, polynucleotides, nucleic acids, and aptamers.

The linker 33 may be a material or substance that can connect and integrate one or a plurality of tags 31 and the probe 32. The linker 33 may contain a material or substances that can also change the properties of one or more tags 31 so that the tags 31 can be identified differently by a measurement method of the tag 31 when the probe 32 has become bound to a target (analyte, object). As one example, it is known that Raman scattering shifts in wavelength when the probe 32 binds to a target due to changes in the overall structure or number of molecules of the molecular sensor 30. Since the probe 32 is designed to bind to a specific target, any change in state after binding, such as the peak position, can be determined during the design of the molecular sensor 30. The linker 33 may include an organic molecular backbone (framework) part, which may be formed from phosphodiester bonds, peptide bonds and/or glycosidic bonds. The backbone part may include nucleotides, amino acids, monosaccharides, or any of a variety of known plastic monomers, such as vinyl, styrene, carbonate, acetate, and acrylamide.

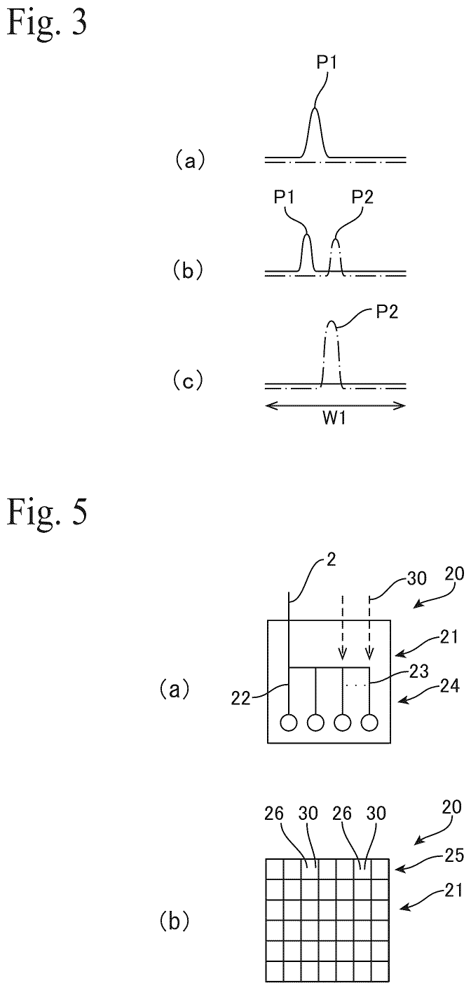

As shown in FIG. 3(a), for example, when the sample 2 does not contain an object to be analyzed (analyte, target component, target), only peak P1 will appear in the measurement results of the sample 2 with the molecular sensor 30, as one example, by mixing the molecular sensor 30 or placing the molecular sensor 30 in contact with the sample 2. If sample 2 does contain a target object at a concentration that is equal to or higher than an expected concentration, there is the possibility that all the molecular sensor 30 contact with the sample 2 will bind to the target. If this happens, as shown in FIG. 3(c), only peak P2 corresponding to a position of the molecular sensor 30 bound to the target will appear. When the sample 2 contains the target with the expected concentration, as shown in FIG. 3(b), both peaks appear, and the peak P1 of the molecular sensor 30 will decrease and the peak P2 of the molecular sensor 30 bound to the target will increase.

Accordingly, even if the target contained in sample 2 is a trace amount or a weak component that is difficult to measure with the same accuracy as other components when a specified measurement method is used, the concentration of the target contained in the sample 2 can be accurately measured by an increase or decrease in the peak P2 corresponding to a part of the molecular sensor 30 bound to the target, and analysis results of the sample 2 can be obtained. The concentration of the target may be analyzed by comparing the increase or decrease in the peak P2 of the molecular sensor 30 that is bound to the target with the increase or decrease in peak P1 of the molecular sensor 30 itself. The peak P1 of the molecular sensor 30 itself does not need to appear in a measurement region, as one example, a detection wavelength region W1. By designing and providing the molecular sensor 30 so that the peak P1 of the molecular sensor 30 itself and the peak P2 of the part of the molecular sensor 30 bound to a target do not overlap a characteristic peak (fingerprint) of the sample 2, it is possible to analyze the components of the sample 2 with even greater accuracy.

The target (object of measurement, object of detection, or analyte substance) of the molecular sensor 30 may be any atom, chemical, molecule, compound, composition, microorganism, or aggregate that is to be measured (detected) and/or identified that may include, for example, but are not limited to, amino acids, peptides, polypeptides, proteins, glycoproteins, lipoproteins, nucleosides, nucleotides, oligonucleotides, nucleic acids, sugars, carbohydrates, oligosaccharides, polysaccharides, fatty acids, lipids, hormones, metabolites, cytokines, chemokines, receptors, neurotransmitters, antigens, allergens, antibodies, substrates, metabolites, cofactors, inhibitors, drugs, pharmaceuticals, nutrients, prions, toxins, poisons, explosives, agricultural chemicals, pesticides, chemical warfare agents, biological hazards, radioisotopes, vitamins, heterocyclic aromatic compounds, carcinogens, mutagens, narcotics, amphetamines, barbiturates, hallucinogens, waste products, and/or pollutants. Such microorganisms include, but are not limited to, viruses, bacteria, and cells.

The information providing system 1 according to the present embodiment includes the system 10 that analyzes an objective material (sample) 2 containing multiple components using the molecular sensor 30 that includes at least one tag 31 that can be uniquely detected by a predetermined measurement method (first measurement method), the probe 32 that targets and binds to a predetermined molecule, metal, or ion, and the linker 33 that links the tags 31 and the probe 32. The analysis system 10 includes: a measurement apparatus 19 that is configured to measure the objective material (sample) 2 using the first measurement method (for example, CARS); a database 11 including structures and optical properties of the components of the plurality of probes 32, the tags 31, and the linker 33 that can connect them, of the molecular sensor 30; a simulator 12 that is configured to virtually synthesize one or more molecular sensors 30 including an arbitrary probe 32, a tag 31, and a linker 33 that can connect them, and output virtual measurement results (detection results) 55 produced by the first measurement method with the virtual molecular sensor virtually synthesized; a molecular sensor providing apparatus (providing apparatus) 13 that is configured to select and provide at least one molecular sensor 30 including one re more probes, one or more tags, and one or more linkers that are suited to analysis of multiple components of the sample 2 based on (from) virtual measurement results 55 of the simulator 12 with the virtual molecular sensors and preliminary measurement results 59 by the measurement apparatus 19; and an analysis apparatus 14 that is configured to actually analyze the multiple components of the sample 2 based on the measurement result 50 of the sample 2 produced by the measurement apparatus 19 using the provided molecular sensor 30.

The database 11 includes information relating to a plurality of probes 32, a plurality of tags 31, and a plurality of linkers 33, as well as information 35 for simulation that includes the spectrum when a molecular sensor that has been synthesized from these elements is measured using a first measurement method, in the present embodiment, CARS. The simulator 12 generates information 55 including virtual measurement results with at least one virtual molecular sensor 30 including at least one probe 32 for at least one of the objects (components) to be analyzed contained in the sample 2 as targets, at least one tag 31 that can be detected at expected concentrations for the targets, and at least one linker 33 capable of connecting these elements. Information 6 including the targets and the expected concentrations for which the molecular sensors 30 are selected may be obtained from trace (weak) components and their concentrations that are predicted from the preliminary measurement results 59, or may be obtained from information 5a on the trace (weak) components of interest provided by the application 5. Information 6 relating to the targets is provided via the providing apparatus 13 which in the present embodiment includes a function for selecting virtual molecular sensors 30, but may also be provided from a control apparatus 18 of the analysis system 10 which controls every function including the simulator 12.

The function 12a of the simulator 12 predicts (hypothesizes, assumes, virtually set) a molecular sensor 30 which should be effective for a given target, an expected concentration, and measurement method and provides virtual results 55 for when measurement is performed using this molecular sensor 30. This function (apparatus or function that provides information for selection purposes) 12a may predict a plurality of molecular sensors 30 for one combination of a target and expected its concentration and provide (simulate) virtual measurement results 55 virtually measured with these molecular sensors 30. A selection apparatus (selector) 13a of the molecular sensor providing apparatus 13 can select a molecular sensor 30 for obtaining a suitable result for measurement of the sample 2 from these provided virtual measurement results 55. The function 12a of the simulator 12 may predict (hypothesize, assume) a combination of a plurality of molecular sensors 30 for multiple targets and generate virtual measurement results 55 for simultaneous measurement by these molecular sensors 30 as results of simulation. A set of a plurality of molecular sensors 30 may be used for a plurality of targets in this simulation and virtual measurement results 55 may be generated for this set of molecular sensors. One example of a virtual measurement result 55 is a CARS spectrum.

Another function 12b of the simulator 12 is a function (learning data generation function) for generating learning data (teacher data, training data) 60 for generating a learned model (trained model, artificial intelligence) 16. This function 12b generates a virtual measurement result 55 by measuring with virtual molecular sensors that is corresponding to the molecular sensor 30 or a set of molecular sensors 30 selected for measurement purposes of the virtual samples uses that include virtual components 56 of expected concentration varied within expected ranges. The virtual components 56 may include the result 59 of the preliminary measurement of a real sample 2. This function 12b generates virtual measurement results (that are, virtual spectra) 55 by simulating with the molecular sensors 30 for a plurality of virtual components 56 that include the virtual concentrations of a target or set of targets of the molecular sensors 30 to be used in the actual measurement are varied. In addition, this function 12b provides a plurality of replicas 60 of measurement results virtually by the simulations, which include a plurality of virtual components 56 and virtual measurement results 55 for each virtual component 56, to a learning apparatus 15 as the learning data.

The molecular sensor providing apparatus 13 includes a function as an automatic design apparatus (automatic designer) for a programmable molecular sensor 30. The molecular sensor providing apparatus 13 includes a function (selector) 13a for selecting a molecular sensor 30 to be used for actual measurement and a function (generator) 13b for automatically generating the selected molecular sensor 30. The selector 13a sets information (that is, information including a predicted target and the predicted concentration) 6 relating to a target or targets including at least one of the weak components that can be predicted from the results of a preliminary measurement of the sample 2 by the measurement apparatus 19 and/or at least one weak component in the object (object of interest) 5a specified by the application 5, and acquires, from the simulator 12, virtual measurement results (detection results) 55 with at least one virtual molecular sensor 30 including probes 32 corresponding to one or a plurality of targets, at least one tag 31 that can be detected at the expected concentration of the target, and at least one linker 33 that can connect these components. The selector 13a further selects or automatically designs at least one molecular sensor 30 including a probe 32, a tag 31 and a linker 33 suited to analyzing multiple components of the sample 2 based on virtual analysis results obtained by combining the virtual measurement results 55 and the preliminary measurement results 59. The generator 13b automatically generates the selected real one or more molecular sensors 30 and prepares it for use in measurement. The generator 13b may provide one or more molecular sensors 30 by selecting them from a variety of stocked molecular sensors 30, for use in actual measurement.

The components (targets) to be measured using the molecular sensors 30 and its expected concentrations may be found from the preliminary measurement results. For example, it is possible to predict the targets and concentrations contained in the sample 2 from weak information which may be similar to noise, such as side lobes, contained in the spectrum of the result 59 produced by preliminary measurement. Additionally, targets and their expected concentrations may be found by the specification of the application 5, which provides information based on data of a specific analytical interest. As one example, if the objective material (sample) 2 is blood, the concentrations in the blood of target trace components and/or of target ions that are difficult to directly measure using the CARS measurement apparatus 19, are varied within a certain range. The same also applies to other applications. A molecular sensor 30 that can be detected by or is suitable for detection by the CARS measurement apparatus 19 can be selected to target a desired weak component contained in the sample 2 and can also be selected so that a signal of the molecular sensor 30 obtained by the CARS measurement apparatus 19 does not interfere with or is easily separated from the signal (spectrum) of the components of the sample 2 that can be directly measured.

Accordingly, within a range where the objective is analysis, the molecular sensor 30 used to measure the sample 2 can be designed so that the measurement system (that is, the CARS measurement apparatus) 19 can obtain an output and a resolution that enable quantitative analysis for a relationship between an existing signal (spectrum) obtained from the sample 2 and another signal (spectrum) obtained with one or more molecular sensors 30. The molecular sensors 30 may be designed in a manner that are suited to an analysis system that analyzes the signal produced by the measurement system 19 used in the analysis apparatus 14. In the case of multivariate analysis, a combination of molecular sensors 30 may be designed that provides sufficient resolution for quantitative analysis by an analysis protocol, and when analysis using a learning model (AI) is used, a combination of molecular sensors 30 may be designed that enables a model for learning to be appropriately created.

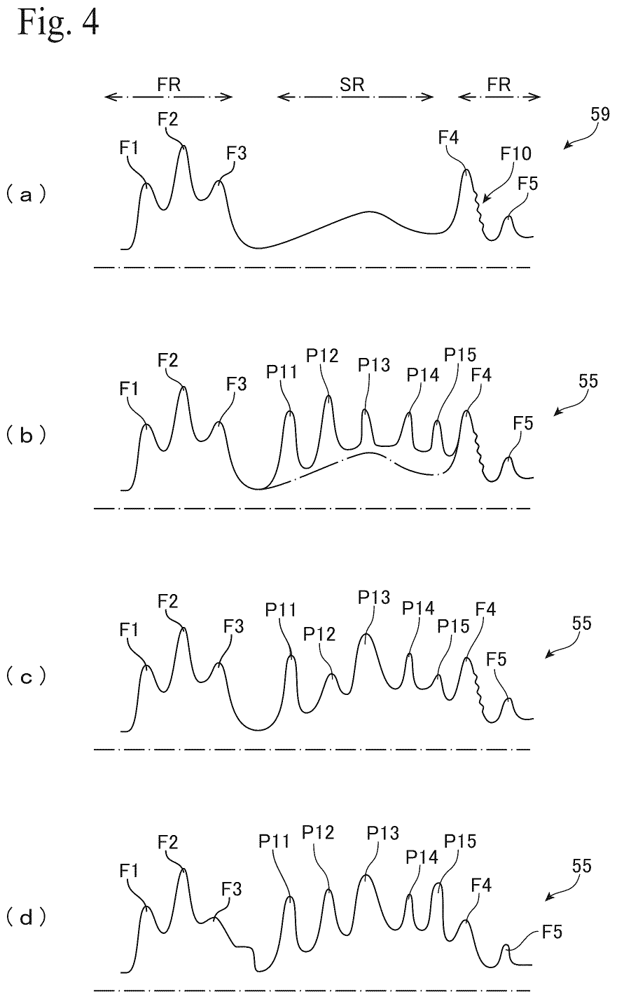

FIG. 4 shows some example measurement results (detection results) used in the analysis system 10. FIG. 4(a) is one example of a preliminary measurement result 59. The result (spectrum) 59 produced by direct measurement of the sample 2 by the CARS measurement apparatus 19 may include a fingerprint region FR, which includes peaks F1 to F5 reflecting several components contained in the sample 2, and a silent region SR, in which hardly any peaks are detected. The result 59 of the preliminary measurement may contain information from components that are present in trace amounts and do not appear as significant peaks or from weak components that are difficult to detect as molecular motion as weak signals in a side lobe F10 or in the silent region SR.

FIG. 4(b) is one example of a virtual measurement result (spectrum) 55 obtained by measuring (simulating) the sample 2 in the simulator 12 with the virtual molecular sensors 30. As one example, by setting up five molecular sensors 30 for five targets and having peaks P11 to P15 indicating their measurement results appear in the silent region SR, the concentration of each target contained in the sample 2 can be measured without interfering with the peaks F1 to F5 in the fingerprint region FR of the sample 2.

FIGS. 4(c) and (d) depict examples of virtual measurement results (that is, simulation results) 55 produced by setting virtual components 56 in the simulator 12 by varying the concentrations of targets contained in the sample 2 and/or by changing the concentrations of components (main components) contained in the sample 2 that can be measured without using the molecular sensor 30 and then measuring such virtually varied components 56 with the molecular sensors 30. By setting a plurality of virtual components 56 by varying the concentrations of targets or varying the concentrations of the main components and obtaining corresponding virtual measurement results 55 through simulation, it is possible to verify in advance the changes in height and position of the peaks P11 to P15 of the molecular sensors 30 together with the changes in height and position of the peaks F1 to F5 in the fingerprint. Accordingly, a trained learning model (learned model) 16 can be generated by machine learning or deep learning of an analytical learning model 16 using replicas 60 of measurement results that include combinations of virtual components 56 and virtual measurement results 55 for such virtual components 56.

The analysis system 10 includes the learning apparatus 15 that generates a trained learning model (learned model, trained model) 16 that outputs (estimates) analysis results from the measurement results 50 using replicas 60 of the measurement results provided by the simulator 12 as learning data (teaching data, training data). The analysis system 10 may further include the learning apparatus 15 that generates a trained learning model (learned model, or trained learning module (AI)) 16 by machine learning of the virtual analysis results 55 of the simulator 12 where at least one selected molecular sensor 30 is used as a virtual molecular sensor for a number of predicted objective materials including different predicted targets and predicted concentrations. The analysis system 10 may be configured to automatically design molecular sensors 30, generate a large number of replicas that have different measurement results using these automatically designed molecular sensors 30, and use these replicas to generate an AI module 16, which has been trained in advance by machine learning and then used in the analysis. The learned model may be generated using various conventional machine-learning techniques as appropriate. As one example, the model may be generated using a machine learning technique for supervised learning, such as support vector machine (SVM). The model may be generated using deep learning techniques. As examples, the model may be generated using various deep learning techniques, such as a deep neural network (DNN), a recurrent neural network (RNN), or a convolutional neural network (CNN).

The analysis apparatus 14 of the analysis system 10 may include an analysis module 14a that analyzes results 55 of actual measurement using the learned model 16. The analysis apparatus 14 may have a function that analyzes information on a multi-component system using other methods, such as a multivariate analysis module, in addition to or alternatively the analysis using the learned model 16. The molecular sensors 30 selected and provided by the molecular sensor providing apparatus 13 may be supplied to the holder 20 together with the sample 2 and measured (detected) by the CARS measurement apparatus 19 in a state where the molecular sensors 30 have been mixed with the sample 2. The molecular sensors 30 may be provided by being fixed to a substrate or chip so that the molecular sensors 30 can contact the sample 2 and enable the CARS measurement apparatus 19 to acquire data relating to the concentration of a target contained in the sample 2, or may be provided in other ways so that targets contained in the sample 2 can bind to the probes 32 of the molecular sensors 30.

For measuring a multi-component sample 2 with one or a plurality of molecular sensors 30, it is desirable to obtain a predetermined resolution when using the molecular sensors 30 simultaneously. On the other hand, simulations may find problems with such measurement methods, such as insufficient resolution. The selector 13a of the molecular sensor providing apparatus 13 may derive the solution to such problems. As one example, the selector 13a may design a measurement method of the measurement apparatus 19 as a program (program product) including a combination of times (that is, a sequence) and molecular sensors 30 to be used with that sequence. As a result, in addition to providing the molecular sensors 30, the molecular sensor providing apparatus 13 may also design or manufacture and provide a cell (holder) 20 that holds the sample 2 for measurement purposes in the measurement apparatus 19 as a chip or flow site that includes a combination of addresses (locations) and molecular sensors 30. The providing apparatus 13 may select (that is, design and provide) the molecular sensors 30 using an appropriate optimization program or a learned model (that is, artificial intelligence) so that the measurement time and resolution of the measurement apparatus 19 are optimized. In addition, factors to be optimized may additionally include the amount of the sample required to perform measurement, deterioration of the sample due to continuous measurement, accuracy, and cost.

The holder 20 in the present embodiment may include an address assignment apparatus 21 that assigns two-dimensional or three-dimensional addresses to the molecular sensors 30 provided by the molecular sensor providing apparatus 13. The measurement apparatus 19 also includes a scanning unit (scanner) 19a for measuring the sample 2 in units of addresses using CARS. Additionally, the analysis apparatus 14 further includes an evaluation unit 14b that evaluates the measurement results acquired in units of addresses.

FIG. 5 shows some examples of automatically designed or automatically provided programmable address assignment apparatuses (multi-address cells) 21. FIG. 5(a) depicts an example of a multi-flow cell 24 equipped with multiple flows. The flow cell 24 includes a path 22 on which the sample 2 flows and is measured without adding a molecular sensor 30 and paths 23 on which one or more molecular sensors 30 are individually injected and placed in contact with the sample 2 before measurement is performed. The flow cell 24 can include various routes, such as a cell in which molecular sensors 30 are sequentially added. The analysis apparatus 14 combines the measurement results from each path (address) and analyzes the components contained in the sample 2. The CARS measurement apparatus 19 can scan the paths 22 and 23 to acquire measurement results for each address, that is, measurement results that do not include a molecular sensor 30 and measurement results that each include different molecular sensors 30. The analysis apparatus 14 can accurately analyze the components of the sample 2 qualitatively and quantitatively, including trace or weak components, from measurement results that do not include a molecular sensor 30 and measurement results that include different molecular sensors 30. Such multi-address cells 21 may be automatically generated, or may be produced by reconfiguring a plurality of flows or paths or reconfiguring tags 31 and probes 32 connected to linkers 33 that are placed in advance at predetermined addresses.

FIG. 5(b) is one example of an address assignment apparatus 21 including a multi-address substrate (chip) 25 equipped with a number of sections 26 that each hold a different molecular sensor 30. Each section 26 may hold one or a plurality of molecular sensors 30 and may further function as a chip for surface-enhanced Raman spectroscopy (SERS) configured with metal particles or the like capable of exciting localized surface plasmon resonance. If the measurement apparatus 19 is a CARS analysis unit, a laser light source is scanned by the scanning unit 19a to obtain measurement results in units of addresses. That is, the position (region, laser spot, or spot) of each section 26 on the surface of the chip 25 can be scanned (irradiated or focused) via the sample 2 with the pump light and Stokes light, which are laser light obtained from the laser light source, to measure the enhanced scattered light in each section 26 of the chip 25 provided for enhancement purposes. The chip 25 on which the molecular sensors 30 are fixed and supported may contain porous glass beads, plastic, polysaccharide, nylon, nitrocellulose, a composite material, ceramic, plastic resin, silica, a silica-based material, silicon, modified silicon, carbon, metal, inorganic glass, a fiber optic bundle, or any other type of conventionally known solid support material.

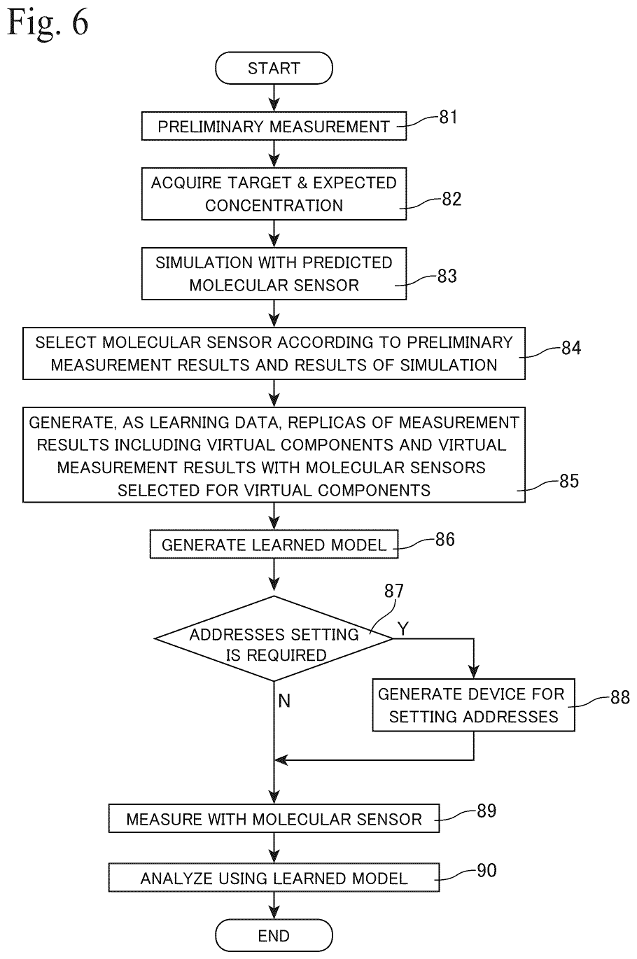

FIG. 6 is a flowchart outlining an analysis method using the analysis system 10. This method may be provided by being recorded on an appropriate recording medium as a program (or program product) 18p to be executed by the control apparatus (controller) 18 that controls the analysis system 10. The control apparatus 18 may be equipped with computer resources, such as a CPU and memory.

First, in step 81, the sample 2 is preliminarily measured by the CARS measurement apparatus 19, and the result 59 is acquired by the providing apparatus 13. In step 82, the providing apparatus 13 generates, based on the preliminary measurement results 59 and/or the subject of interest of the application 5, information 6 relating to targets and their expected concentrations for preparing one or more molecular sensors 30, and in step 83, the simulator 12 performs a simulation with the predicted (assumed, hypothesized) one or more molecular sensors 30 (as virtual molecular sensors) and generates virtual measurement results for the virtual molecular sensors (if the virtual molecular sensors are applied). In step 84, a selection unit (selector) 13 of the providing apparatus 13 selects one or more molecular sensors 30 to be used for measurement based on the preliminary measurement results 59 and virtual measurement results 55 obtained from the simulator 12. In step 85, the simulator 12 generates, as learning data, replicas 60 of measurement results including the virtual components 56 and virtual measurement results 55 using the molecular sensors 30 selected (as a virtual molecular sensor) for such virtual components 56.

In step 86, the learning apparatus 15 uses the replicas 60 as the learning data to perform machine learning of a learning model to generate a trained learning model (learned model) 16. In step 87, the providing apparatus 13 determines whether it is necessary to set addresses, that is, whether an address assignment apparatus 21 is required. If required, in step 88, the providing apparatus 13 generates and provides a device for address assignment (that is, address setting). In step 89, the CARS measurement apparatus 19 measures the sample 2 using one or more molecular sensors 30. In step 90, the analysis apparatus 14 uses the learned model 16 to provide results of analyzing the sample 2 based on the measurement results 50 of the CARS measurement apparatus 19.

Although one example that uses CARS has been described above as an example of Raman spectroscopy as a measurement method (first measurement method) including detection of tags 31, any suitable type or configuration of Raman spectroscopy or related technology that is conventionally known may be used, with examples including standard Raman scattering, resonance Raman scattering, surface-enhanced resonance Raman scattering (SERS), tip-enhanced Raman scattering (TERS), coherent anti-Stokes Raman spectroscopy (CARS), stimulated Raman scattering (SAS), inverse Raman spectroscopy, stimulated gain Raman spectroscopy, hyper-Raman scattering, molecular optical laser examiner (MOLE), Raman microprobe, Raman microscopy, confocal Raman micro spectrometry, 3D or scanning Raman, Raman saturation spectroscopy, time-resolved resonance Raman spectroscopy, Raman decoupling spectroscopy or UV-Raman microscopy.

In addition, the present invention can be used with other suitable imaging modalities, for example, by using tags that can be read by any of fluorescence microscopy, FTIR (Fourier Transform Infrared) spectroscopy, Raman spectroscopy, electron microscopy, and surface plasmon resonance. The molecular sensor 30 may be any sensor where a tag 31 becomes activated or inactivated for the first measurement method or the state detected by the first measurement method changes when the probe 32 binds to a target, and the molecular sensor 30 may be selected depending on the properties of the signal detected in the first measurement method. Example tags may include fluorescent tags, Raman tags, nanoparticle tags, nanotube tags, fullerene tags or quantum dot tags.

The above description discloses a method for analyzing an objective material (system) containing multiple components using one or more molecular sensors including at least one tag that can be uniquely detected by a first measurement method, a probe that is configured to bind to a specific molecule, metal, or ion, as a target and a linker that links the at least one tag and the probe, the method including:
(a) performing preliminary measurement by the first measurement method of the objective material;
(b) using a simulator that virtually synthesizes a molecular sensor including a (an arbitrary, a freely chosen) probe and a (an arbitrary, a freely chosen) tag, and a linker that can connect them and outputs virtual measurement results (virtual detection results) of the first measurement method with such virtual molecular sensor or virtual molecular sensors to select and automatically generating at least one molecular sensor including a probe, tag, and linker suited to analysis of the multiple components of the objective material based on a virtual analysis results produced by combining the preliminary measurement results and the virtual measurement results with at least one virtual molecular sensor, wherein the at least one virtual molecular sensor includes at least one probe for predicted at least one target including at least one trace or weak component that can be predicted from the preliminary measurement and/or at least one trace or weak component of the objective material that has been specified by an application, at least one tag which can be detected at expected concentrations of the at least one target and at least one linker capable of connecting them;
(c) actual analyzing the multiple components of the objective material by the first measurement method by setting or placing the at least one molecular sensor automatically generated in contact with the objective material.

The method may include generating a learning module (learning model, learned model) by performing machine learning on the virtual analysis results of the simulator using at least one selected molecular sensor as a virtual molecular sensor for a number of predicted objective materials including different predicted target components and predicted concentrations, and the actual analyzing may include analyzing the measurement results using such learning module. The at least one automatically generated molecular sensor may each have a linker with a two-dimensional or three-dimensional address, and in the method, the analysis may include measuring the objective material using the first measurement method in units of addresses. The first measurement method may include CARS and the tags may include Raman tags. The linker may include an organic molecular backbone (framework) and the molecular sensor may be a biosensor.

The above description also discloses a system for analyzing an objective material (target system) containing multiple components using one or more molecular sensors including at least one tag that can be uniquely detected by a first measurement method, a probe that is configured to bind to a specific molecule, metal, or ion, as a target and a linker that links the at least one tag and the probe. This system includes:
(a) a measurement apparatus that measures the objective material according to the first measurement method;
(b) a database including structures and optical properties related to a plurality of probes, a plurality of tags, and a plurality of linkers that can connect them each other, and a simulator that virtually synthesizes a molecular sensor including a (an arbitrary, a freely chosen) probe and a tag and a linker that can connect them and outputs virtual measurement results of by the first measurement method with such virtual molecular sensors;
(c) a molecular sensor generating apparatus that selects and automatically generates at least one molecular sensor including a probe, a tag, and a linker suited to analysis of the multiple components of the objective material from a virtual analysis result produced by combining the preliminary measurement results and the virtual measurement results with at least one virtual molecular sensor, wherein the at least one virtual molecular sensor includes at least one probe for predicted at least one target including at least one trace or weak component that can be predicted from the preliminary measurement results of the objective material by the measurement apparatus and/or at least one trace or weak component of an objective material that has been specified by an application, at least one tag which can be detected at an expected concentration of the at least one target, and at least one linker capable of connecting them; and
(d) an analysis apparatus that performs actual analysis of the multiple components of the objective material (target system) based on a measurement result by the measurement apparatus where the at least one molecular sensor automatically generated is placed in contact with the objective material.

The system may include a learning apparatus that generates a learning module (learned model) by performing machine learning on the virtual analysis results of the simulator using (with) at least one selected molecular sensor as a virtual molecular sensor for a number of predicted objective material including different predicted target components and predicted concentrations, and the analysis apparatus may include the learned module for analyzing the measurement results. The analysis apparatus may provide the linker of each of the at least one automatically generated molecular sensors with a two-dimensional or three-dimensional address, and the measurement apparatus may measure the target system using the first measurement method in units of addresses.

Note that although specific embodiments of the present invention have been described above, various other embodiments and modifications will be conceivable to those of skill in the art without departing from the scope and spirit of the invention. Such other embodiments and modifications are addressed by the scope of the patent claims given below, and the present invention is defined by the scope of these patent claims.

1. A system comprising:
a measurement apparatus that is configured to measure an object in a sample using a first measurement method;
a database that stores information including structures and optical properties relating to a plurality of probes, a plurality of tags, and a plurality of linkers capable of connecting any of the plurality of probes and any of the plurality of tags;
a simulator that is configured to virtually synthesize a molecular sensor including at least one tag that is able to be uniquely detected by the first measurement method out of the plurality of tags, an arbitrary probe, and a linker capable of connecting the at least one tag and the probe, and obtain a virtual measurement result of the first measurement method with a virtual molecular sensor synthesized virtually;
a molecular sensor providing apparatus that is configured to select and provide at least one molecular sensor including a probe, a tag, and a linker that are suited to analyzing multiple components including a target contained in the sample based on preliminary measurement results of the sample by the measurement apparatus and virtual measurement results by the simulator with at least one virtual molecular sensor, wherein the at least one virtual molecular sensor includes:
at least one probe for at least one of the multiple components of the object in the sample as the target;
at least one tag that is detectable at an expected concentration of the target; and
at least one linker capable of connecting the at least one probe and the at least one tag, and
an analysis apparatus that is configured to analyze components of the sample based on measurement results of the sample by the measurement apparatus with the at least one molecular sensor provided by the molecular sensor providing apparatus.

2. The system according to claim 1,
further comprising a learning apparatus that is configured to generates a learned model that has been trained using learning data that includes replicas of a plurality of measurement results including the virtual measurement results of the simulator with the at least one molecular sensor provided by the molecular sensor providing apparatus as the virtual molecular sensor for a plurality of virtual samples with a plurality of virtual components which include the target and have different expected concentrations,
wherein the analysis apparatus includes an analysis module that is configured to analyze the measurement results by the learned model.

3. The system according to claim 1,
wherein the target of the molecular sensor providing apparatus includes at least one of weak components predicted from the preliminary measurement results and a weak component of interest.

4. The system according to claim 1,
further comprising an address assignment apparatus that is configured to assign a two-dimensional or three-dimensional address to the at least one molecular sensor provided by the molecular sensor providing apparatus,
wherein the measurement apparatus is configured to measure the sample in units of addresses using the first measurement method.

5. The system according to claim 1,
wherein the tags include Raman tags.

6. The system according to claim 1,
wherein the linker includes an organic molecular backbone.

7. The system according to claim 1,
wherein the first measurement method includes any of standard Raman scattering, resonance Raman scattering, surface-enhanced resonance Raman scattering (SERS), tip-enhanced Raman scattering (TERS), coherent anti-Stokes Raman spectroscopy (CARS), stimulated Raman scattering (SAS), inverse Raman spectroscopy, stimulated gain Raman spectroscopy, hyper-Raman scattering, molecular optical laser examiner (MOLE), Raman microprobe, Raman microscopy, confocal Raman micro spectrometry, three-dimensional scanning Raman, Raman saturation spectroscopy, time-resolved resonance Raman, Raman decoupling spectroscopy, and UV-Raman microscopy.

8. A method for performing analysis with a system including a measurement apparatus that measures an object for analysis of a sample according to a first measurement method,
wherein the system includes: a database that stores information including structures and optical properties relating to a plurality of probes, a plurality of tags, and a plurality of linkers capable of connecting any of the plurality of probes and any of the plurality of tags; and
a simulator that virtually synthesizes a molecular sensor including at least one tag that is able to be uniquely detected by the first measurement method out of the plurality of tags, an arbitrary probe, and a linker capable of connecting the tag and the probe, and obtains a virtual measurement result of the first measurement method with a virtual molecular sensor synthesized virtually,
the method comprising:
obtaining virtual measurement results by the simulator with at least one virtual molecular sensor including at least one probe for at least one of components of the object contained in the sample as a target, at least one tag detectable at an expected concentration of the target, and at least one linker capable of connecting the probe and the tag;
selecting at least one molecular sensor which includes a probe, a tag, and a linker that are suited to analyzing multiple components including the target contained in the sample based on the virtual measurement results and preliminary measurement results of the sample by the measurement apparatus; and
analyzing components of the sample based on measurement results of the sample by the measurement apparatus with the at least one selected molecular sensor.

9. The method according to claim 8, further comprising:
generating replicas of a plurality of measurement results including the virtual measurement results of the simulator with the virtual molecular sensor, which is the at least one molecular sensor selected, for a plurality of virtual samples with a plurality of virtual components which include the target and have different expected concentrations, and
generating a learned model by performing machine learning with the replicas of the plurality of measurement results as learning data,
wherein the analyzing includes analyzing the measurement results using the learned model.

10. The method according to claim 8,
wherein the target includes at least one of weak components predicted from the preliminary measurement results and a weak component of interest.

11. The method according to claim 8,
further comprising assigning a two-dimensional or three-dimensional address to the selected at least one molecular sensor,
wherein the analyzing includes measuring by the measurement apparatus the sample in units of addresses.

12. A control program of a system including a measurement apparatus that measures an object for analysis of a sample using a first measurement method,
wherein the system includes: a database that stores information including structures and optical properties relating to a plurality of probes, a plurality of tags, and a plurality of linkers capable of connecting any of the plurality of probes and any of the plurality of tags; and
a simulator that virtually synthesizes a molecular sensor including at least one tag that is able to be uniquely detected by the first measurement method among the plurality of tags, an arbitrary probe, and a linker capable of connecting the tag and the probe, and obtains a virtual measurement result of the first measurement method with a virtual molecular sensor synthesized virtually,
the program comprising instructions that cause a control apparatus of the system to execute:
obtaining virtual measurement results by the simulator with at least one virtual molecular sensor including at least one probe for at least one of the components of the object contained in the sample as a target, at least one tag detectable at an expected concentration of the target, and at least one linker capable of connecting the probe and the tag;
selecting at least one molecular sensor which includes a probe, a tag, and a linker that are suited to analyzing multiple components including the target contained in the sample based on the virtual measurement results and preliminary measurement results of the sample by the measurement apparatus; and
analyzing components of the sample based on measurement results of the sample by the measurement apparatus with the at least one selected molecular sensor.
